(19) United States
(12) Patent Application Publication
(10) Pub. No.: US 2004/0138729 A1
(43) Pub. Date: Jul. 15, 2004

(54) Head area heat exchange apparel and system

(76) Inventor: Andrea Ladmer, El Cajon, CA (US)

Correspondence Address: BROWN, MARTIN, HALLER & MCCLAIN LLP, 1660 UNION STREET, SAN DIEGO, CA, 92101-2926, US

(21) Appl. No.: 10/439,027

(22) Filed: May 14, 2003

Related U.S. Application Data
(60) Provisional application No. 60/439,604, filed on Jan. 10, 2003.

(51) Int. Cl. A61F 7/00

(52) U.S. Cl. 607/109; 607/112

(57) ABSTRACT

A heat exchange garment or a system is worn on a user's head for engaging the head area and having extensions for surrounding the neck. The garment comprises an inner layer and an outer layer. Substantially, the entire volume between the inner layer and the outer layer may be filled with heat exchange material. Alternatively, sections may be formed for retaining individual thermal packs, each in registration with a portion of the head area or neck.

### CROSS REFERENCE TO RELATED APPLICATIONS

This application claims the benefit of priority of Provisional Patent Application No. 60/439,604 filed Jan. 10, 2003.

### BACKGROUND OF THE INVENTION

The present invention relates to apparel and a system for thermal application to the entire head, ears, eyes, cheek, jaws, chin, and neck area or a selected portion or portions thereof.

Thermal applicators perform heat exchange by heating or cooling a portion or portions of the body. Heat therapy is used in connection with some conditions while cooling therapy is used for others. For example, certain sinus ailments require heat treatments applied as close as possible to sinus cavities. Swelling following medical surgery, dental surgery or other trauma generally requires the application of cold. The present invention addresses thermal therapy to the head area, which includes the head or neck or to a selected portion or portions thereof.

Various devices have been provided for thermal therapy in the head area for more than the past 100 years. Despite the plethora of available devices, when advising patients who have swelling in the area of sutures from recent surgeries, it is extremely common for surgeons to advise patients to apply a bag of frozen peas. Application of frozen peas represents an attractive alternative to an ice pack since peas within the bag are readily moldable about a targeted body region. Peas also exhibit good specific heat characteristics and have a lower freezing point than that of water. A common therapy for treatment of injury is to apply the ice pack for 20 to 30 minutes at a time. Bags of frozen peas cannot be used in a “hands free” mode. A patient normally must hold the ice pack to the affected area and generally can do little else but sit or otherwise remain motionless.

Prior art devices include masks which can be tied or strapped to the face. A mask may represent an unbalanced weight on the face and tend to move unless tightly tied or affixed by uncomfortable elastic securing means. Discomfort is a negative incentive for a user to keep the cooling device in place for a therapeutic length of time. In application it is desired to apply cold to a number of different parts of the neck or head; a mask may not be configurable to reach all desired areas. It may be necessary to use a number of devices at one time.

Thermal therapy devices are normally unsuited for wearing to bed. A user may desire to have cold applied to relieve pain to enable falling asleep. It does not suit the user's purpose if the device shifts in position or must be removed when a user falls asleep.

It is highly desirable to provide a system in which efficient use of cold or heat therapy can be applied to optimize therapeutic effect versus effort required by the user to achieve the therapeutic effect. This optimization may result in a more expedient healing and recovery time, reduces swelling, pain and discomfort generally associated with medical or dental procedures or traumas in a shorter amount of time. It is also important to maximize the efficacy of a thermal applicator for treatment of patients who cannot tolerate pain medication or patients who prefer to employ holistic medicine.

Prior art devices exist which can be supported to a head. Removable hot or cold packs can be affixed to selected portions of the devices. However, the structure provides a minimal amount of flexibility in location and choice of type of heat applicator, and does not allow for maximally effective heat transfer through the device to the user. Also, prior art devices do not disclose neck treatment sections integral with a head supported device and effectively interacting with the device. It is also highly desirable to have a system including components which may be all utilized or selectively utilized on a patient.

### SUMMARY OF THE INVENTION

It is therefore a particular advantage of the present invention to provide head area heat exchange apparel worn on the head and including cooperating portions for heat exchange with the neck.

It is also a particular advantage in one form to provide apparel of the type described in a one piece device for comfortable hands-free delivery of thermal therapy to the head area.

It is a further particular advantage of the invention in one form to provide apparel worn on the head and comprising chambers each for housing heat exchange materials selectively in registration with a predetermined portion of the head area.

It is a further particular advantage of the present invention in one form to provide a system incorporating a garment of the type described wherein said garment comprises a system having removable sections, individually engageable with a selected portion, for example the ear, or portions of the head area.

Briefly stated, in accordance with the present invention, there are provided heat exchange apparel and a system worn on a user's head for engaging the head area and having extensions for surrounding the neck. The garment comprises an inner layer and an outer layer. Substantially, the entire volume between the inner layer and the outer layer may be filled with heat exchange material. Alternatively, sections may be formed for maintaining discrete heat exchange devices, each comprising heat exchange material in registration with a selected portion or portions of the head area. The sections may each be elastomeric.

### BRIEF DESCRIPTION OF THE DRAWINGS

The invention, both as to its organization and manner of operation may be further understood by reference to the following description taken in connection with the following drawings.

Of the drawings:

FIGS. 1 and 2 are a front and side elevation of a user wearing apparel constructed in accordance with the present invention;

FIG. 3 is a side elevation similar to that of FIG. 2 illustrating an embodiment in which sections are formed in the apparel;

FIGS. 4 and 5 are each a front elevation of an embodiment of the garment illustrated in FIG. 3 respectively including and not including a section covering the upper face;

FIGS. 6a, 6b and 6c are each an illustration of a thermal pack suitable for retaining in one section in the embodiment of FIG. 3; and

FIGS. 7 and 8 illustrate a separately wearable thermal pack on and off of an ear of a user respectively.

### DETAILED DESCRIPTION

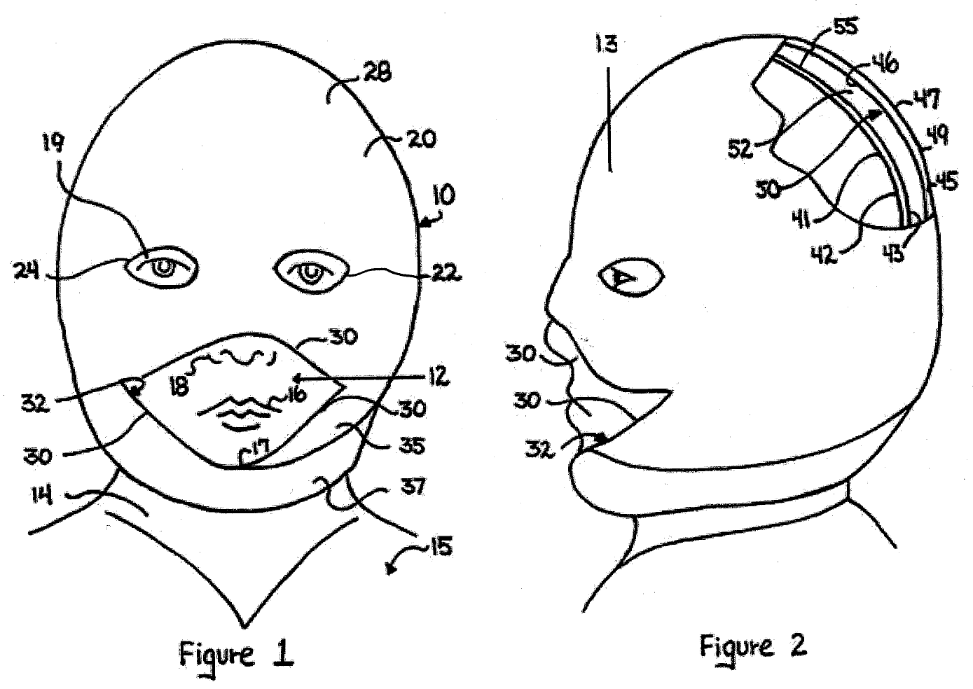

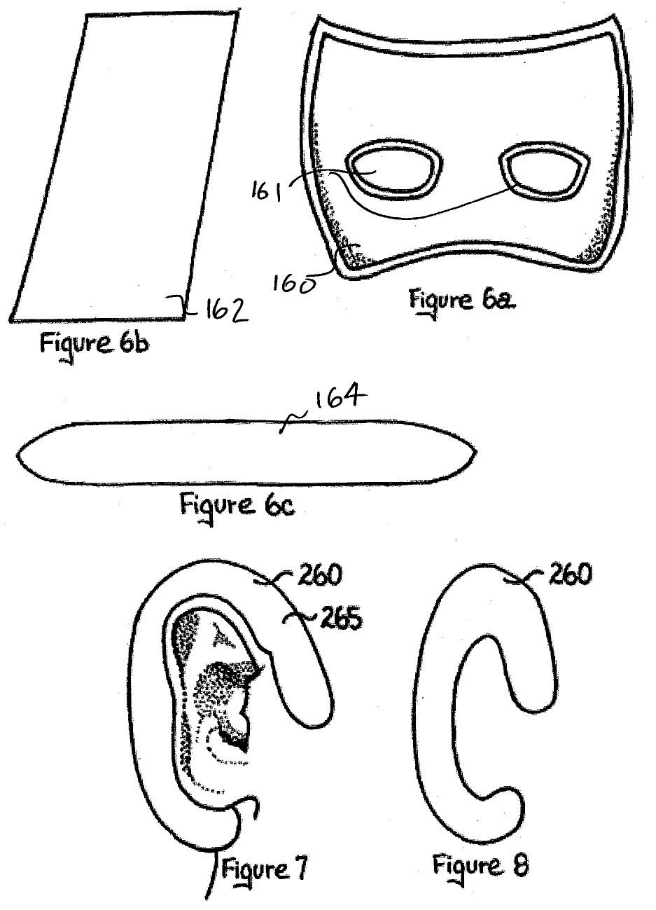

FIGS. 1 and 2 are respectively a front and side elevation of a thermal therapy garment 10 worn by a user 15. The user 15 has an ear 11 (FIG. 7), face 12, head 13, neck 14, mouth 16, chin 17, nose 18 and eyes 19. The garment 10 covers a forehead 21. The garment 10 comprises head cover 28 for generally surrounding a head and may include eye holes 22 and 24. The head cover 28 includes support means 20 which distributes the weight of the garment 10 over the head 13 of the user 15. The support means 20 does not have any particular boundaries within the head cover 28, and in preferred embodiments will not comprise a separate component. The support means 20 may include all portions of head cover 28 transmitting force to the head 13. In one alternative, head cover 28 need not be continuous across the top of the head 13; the support means 20 could comprise strips traversing an opening in the top of head cover 28. Since the weight of the garment 10 is well-distributed, effective centers of gravity of front or rear portions thereof will not tend to exert a force moment about the top of the head 13 of the user 15. This provides for maximum stability of the garment 10 when it is in place and for comfort of the user 15.

An aperture border 30 is provided comprising edges of the garment 10 that will define an outline of an opening 32 when the garment 10 is in an engaged position wrapped around a user 15. The head cover 28 has first and second extensions 35 and 37 which are elongated portions in their unwrapped state. Each of the extensions 35 and 37 depends from one side of the head cover 28 and is preferably deployed by crossing it over the chin 17 and around to the opposite side from which it depends around the neck 14. The extension 37 is similarly deployed. In the embodiment illustrated, a transversely extending border, (the transverse direction being across the face 12 of the user 15) approximately follows the upper extent of the maxilla. The extensions 35 and 37 provide a preferred form of closing the head cover 28. Fasteners or other means may also be used. Additionally, a thermal gel strip may be provided for positioning over a lip and below a nose 14 of the user 15 secured at either end to the head cover 28.

For the purpose of orientation and relative location, locations of portions of the garment 10 are denoted by reference to skull bones with which they are in registration.

The internal structure of the garment 10 is further illustrated with respect to the cross sectional view in the partially broken away portion of FIG. 2. In the description of these components, the terms inner and outer are used. This nomenclature is arbitrary. Within the context of the present description, “inner” will simply be used to mean closer to the head 13, and “outer” will be used to mean farther from the head 13. The garment 10 has an inner layer 41 which engages the head of the user 15. The inner layer 41 has an inner surface 42 and an outer surface 43. The inner layer 41 may be unitary. The garment 10 further comprises an outer layer 45 having an inner surface 46 and an outer surface 47. Additionally, the outer surface 47 may be covered by a head covering layer 49.

In order to provide for heat exchange, a thermal layer 50 is formed between the inner layer 41 and the outer layer 45. The thermal layer 50 includes a thermal element 52. The thermal element 52 may comprise, for example, well-known cooling gel traditionally comprising a starch-based composition and more recently including polymer-based formulations. One such gel is provided under trademark Blue Ice. For heat application, many well known alternatives exist. A new form of rechargeable heat source is sodium acetate with a metal snap disk. Other forms of heated solutions can be microwaved. In this embodiment, the outer surface 43 and inner surface 46 of the inner and outer layers 41 and 45 are impervious. Alternatively, the thermal element 52 may comprise a separately insertable component. Fasteners or stitches 55 may be provided to limit relative movement of the inner layer 41 and the outer layer 45.

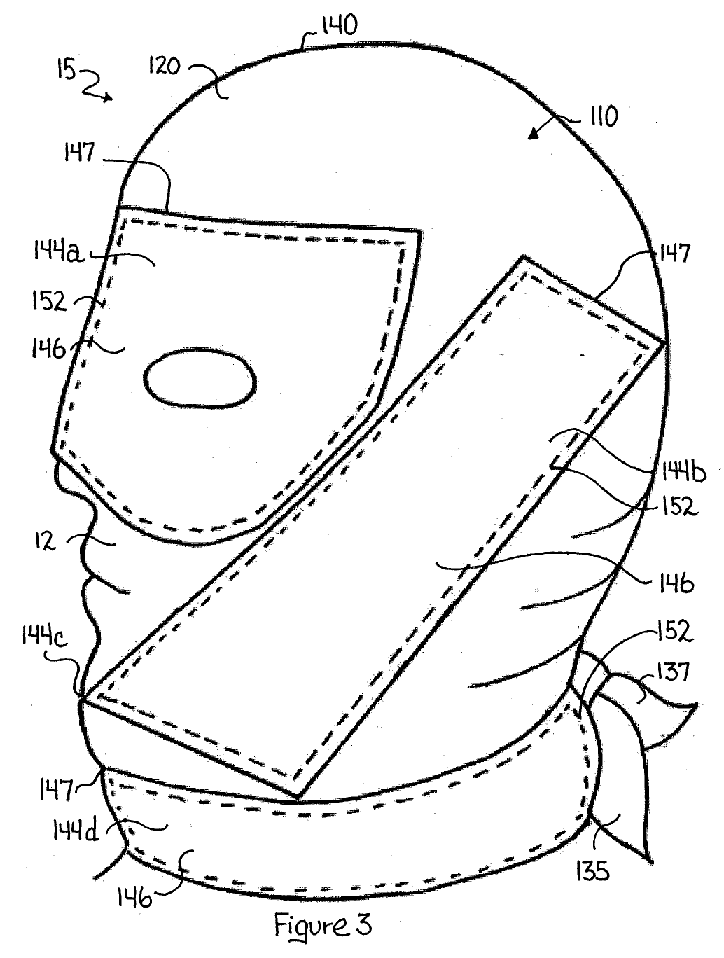

FIG. 3 is a side elevation of a user 15 wearing a further embodiment of the present invention. In this embodiment, a garment 110 is provided comprising a head cover 120. The head cover 120 comprises a layer 140, which is a stretchable textile. Other materials may be used, but a textile will provide greater comfort due to its breathability. Stretchability aids in comfort in fit. The head cover 120 has a plurality of pouches collectively referenced to as 144 formed thereon. For example, a pouch 144a may be provided for covering an upper face area. The upper face area comprises the forehead, eyebrows, eyes, cheeks and nose. A pouch 144b may be provided in the head cover 120 in registration with the mandible and an ear on one side of the face 12. A pouch 144c may cover a corresponding area on an opposite side of the face. Pouch 144d may be inserted into the neck extensions 135 and 137. Each of the pouches 144 may be affixed to an interior or exterior of the layer 140. For greatest convenience, the pouches 144 may be affixed to the exterior of the layer 140. The pouches 144b and 144c may be rectangular, trapezoidal or take other forms.

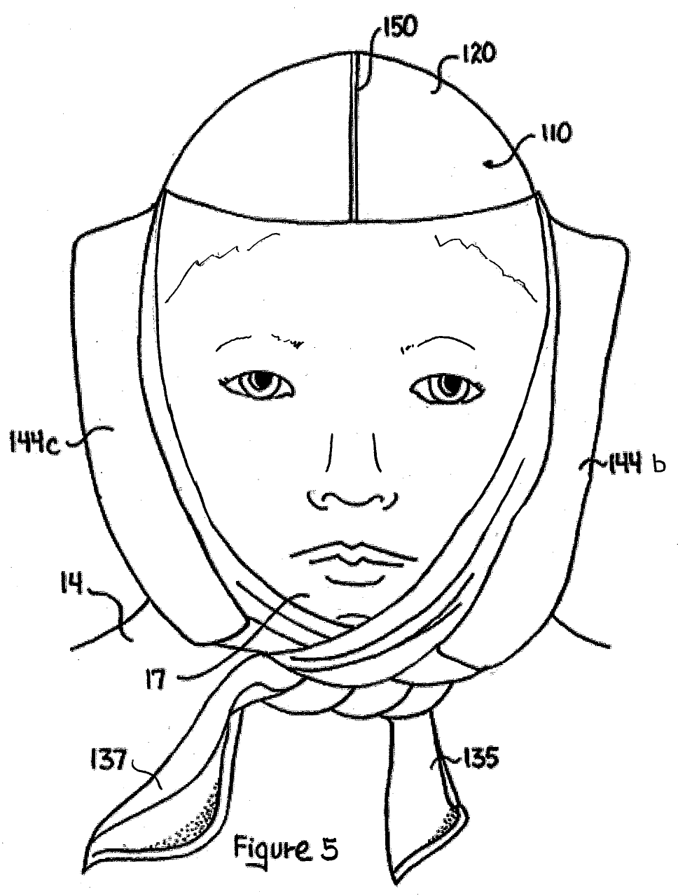

The pouches may each contain a thermal pack as further explained with respect to FIG. 6 below. Additionally, each pouch 144 may be formed with a thermally insulating layer 152 to provide thermal insulation between a thermal pack and the exterior of the garment 110. In a further embodiment, the pouches 144 and the head cover 120 are made of an elastic textile material such as spandex. The spandex is deformable to receive a wide variety of thermal packs. Such a deformable pocket 144 may even utilize the traditional bag of frozen peas as a thermal pack.

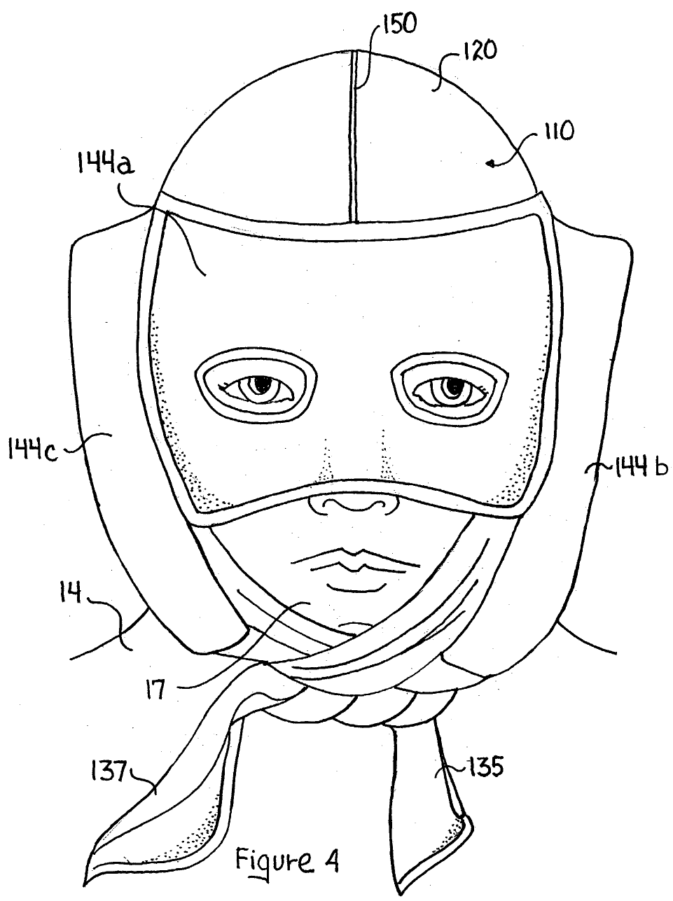

FIGS. 4 and 5 are each a front elevation of an embodiment of the garment illustrated in FIG. 3 respectively including and not including a section covering the upper face. In FIGS. 4 and 5, the head cover 120 is formed in two sections joined by a seam 150. As illustrated in FIG. 3, a thermal pack 160 (see FIG. 6) is included in the pouch 144d. In the exemplifications illustrated in FIGS. 4 and 5, the neck extensions 135 and 137 are tied beneath the chin 17 rather than being wrapped around the neck 14. In this case, the pouch 144d is left empty. Each of the pouches 144 may include a first head covering section 146 for covering a majority of the pouch 144 and having an edge 147 defining an aperture for receiving a thermal pack 160, 162 or 164 (FIG. 6).

FIG. 6 consists of FIGS. 6a, 6b and 6c. FIG. 6a illustrates a thermal pack 160 with eyeholes 161 formed therein that is dimensioned so that it can fit in one of a plurality of the pockets 144 (FIG. 3), and is particularly suited for mounting in the pouch 144a. FIG. 6b represents a thermal pack 162 that is dimensioned to conform to the contour of pouch 144b or pouch 144c (FIG. 3). FIG. 6c illustrates a thermal pack 164 that can be inserted at a midpoint of the thermal garment 110 at an aperture 147 (FIG. 3) and extend into neck extensions 135 and 137. FIGS. 6a through 6c are not to scale.

FIGS. 7 and 8 illustrate a thermal pack 260 which may be included in a kit with a thermal garment according to the present invention. FIGS. 7 and 8 respectively illustrate the thermal pack 260 on and off the ear 11. The thermal pack 260 is substantially C-shaped, and includes a hook portion 265 to rest on the ear 11. The thermal pack 260 may be worn under a thermal garment or may be worn alone.

The specification has been written with a view toward enabling those skilled in the art to make many modifications in the specific examples disclosed to provide a garment and system in accordance with the present invention.

What is claimed is:

1. A thermal garment comprising;
support means to support said garment to a user's head;
said garment having portions to lie in registration with areas on a user to receive thermal therapy;
at least one elongated scarf-like projection depending from said support means having a length sufficient for wrapping around a user's chin and neck;
said garment having an inner surface for contacting a user, said inner surface being thermally conductive, and an outer surface;
said garment comprising a thermal reservoir between said inner surface and said outer surface.

2. A thermal garment according to claim 1 further comprising eyeholes.

3. The thermal garment of claim 2 comprising first and second elongated scarf projections.

4. A thermal garment according to claim 3 wherein said thermal reservoir comprises substantially the entire volume between said inner and said outer layer.

5. A thermal garment according to claim 3 wherein said thermal reservoir comprises at least one pocket for receiving a thermal pack.

6. The thermal garment according to claim 5 wherein each said pocket comprises elastic material.

7. The thermal garment according to claim 6 wherein each said pocket comprises an opening to receive a pack supported in each said pocket.

8. The thermal garment according to claim 7 further comprising a thermal pack in each said pouch.

9. A thermal garment comprising;
support means to support said garment to a user's head;
said garment having portions to lie in registration with areas on a user to receive thermal therapy;
said garment comprising a portion to cover an upper face of a user;
said garment having an inner surface for contacting a user, said inner surface being thermally conductive, and an outer surface;
said garment comprising a thermal reservoir between said inner surface and said outer surface.

10. The thermal garment of claim 9 further comprising eyeholes.

11. A thermal garment according to claim 10 wherein said thermal reservoir comprises substantially the entire volume between said inner and said outer layer.

12. A thermal garment according to claim 10 wherein said thermal reservoir comprises at least one pocket for receiving a thermal pack.

13. A thermal garment according to claim 12 wherein each said pocket elastic material.

14. The thermal garment according to claim 13 wherein each said pocket comprises an opening to receive a pack supported in each said pocket.

15. The thermal garment according to claim 14 further comprising a thermal pack in each said pouch.

16. The thermal garment according to claim 15 further comprising fastening means for closing the thermal garment.

17. A system comprising a head garment covering a portion of a head area and comprising pockets in each in registration with a selected portion of the head, and a removable pouch to be supported in each said pocket, each said pocket comprises means for supporting said pouches as are inserted in said pockets to a user's head.

18. The garment according to claim 17 further comprising a thermal reservoir pouch shaped for reception in each said pocket.

19. A system comprising a head garment covering a head area and comprising pockets each in registration with a selected portion of the head, each pocket for supporting a thermal pack, and further comprising a separately supportable thermal pack shaped to be in registration with a particular area of the head, said thermal pack being formed to be separately supportable on a portion of a user's head apart from said garment.

20. A separately supportable thermal pack according to claim 19 comprising a thermal pack of substantially circular cross section and comprising a substantially C-shaped form to be hooked on a user's ear.

21. A separately supportable thermal pack shaped to be in registration with a particular area of the head, said thermal pack being formed to be separately supportable on a portion of a user's head apart from said garment and comprising a thermal pack of substantially circular cross section and comprising a substantially C-shaped form to be hooked on a user's ear.
